(19) United States
(12) Patent Application Publication
(10) Pub. No.: US 2026/0076692 A1
(43) Pub. Date: Mar. 19, 2026

(54) MEDICAL DEVICE

(71) Applicant: ASAHI INTECC CO., LTD., Seto-shi (JP)

(72) Inventor: Yamato Hirata, Seto-shi (JP)

(73) Assignee: ASAHI INTECC CO., LTD., Seto-shi (JP)

(21) Appl. No.: 19/331,552

(22) Filed: Sep. 17, 2025

(30) Foreign Application Priority Data
Sep. 18, 2024 (JP) 2024-160686

(51) Int. Cl. A61B 17/22; A61B 17/00; A61M 25/09

(52) U.S. Cl. CPC A61B 17/22; A61B 17/00234; A61M 25/09; A61B 2017/00238; A61B 2017/00292; A61B 2017/22042; A61B 2017/22094; A61M 2025/09083; A61M 2025/09175

(57) ABSTRACT

A medical device includes an elongated main body part and a leading portion that is connected to a distal end of the main body part and can enter a lesion. The main body part and the leading portion include a wire. The main body part includes a first portion of the wire, and a second portion of the wire that is different from the first portion and that has a portion facing the first portion in a first direction intersecting an axial direction of the main body part. The leading portion includes a third portion of the wire that is different from the first and second portions and is located between the first portion and the second portion of the wire. A surface of the first portion facing the second portion is a curved surface. A surface of the second portion facing the first portion is a planar surface.

### CROSS-REFEENCE TO RELATED APPLICATIONS

This application claims the benefit of JP 2024-160686, filed Sep. 18, 2024. The disclosure of the prior application is hereby incorporated by reference herein in its entirety.

### TECHNICAL FIELD

The technology disclosed in this specification relates to a medical device.

### BACKGROUND ART

Known medical devices have a main body part and a leading portion. The main body part includes a first wire (first portion), which is a portion of the wire, and a second wire (second portion), which is another portion of the wire and has a portion facing the first wire in a direction intersecting an axial direction of the main body part. The leading portion includes a loop that is another portion of the wire and is located between the first wire and the second wire of the wire (for example, see Patent Document 1).

### PRIOR ART DOCUMENT

### Patent Document

Patent Document 1: Japanese Translation of PCT International Publication No. 2008-514358

### SUMMARY

### Technical Problem

In the above-described medical device, in a portion where the first portion and the second portion face each other, a position of the first portion and a position of the second portion are not easily determined.

The present specification discloses a technology capable of solving the above-described problem.

### Solution to Problem

The technology disclosed in this specification can be achieved, for example, as the following aspects.

A medical device disclosed in this specification includes an elongated main body part, and a leading portion that is connected to a distal end of the main body part and can enter a lesion. The main body part and the leading portion include a wire. The main body part includes a first portion of the wire, and a second portion of the wire that is different from the first portion and that has a portion facing the first portion in a first direction intersecting an axial direction of the main body part. The leading portion includes a third portion of the wire that is different from the first portion and the second portion and is located between the first portion and the second portion of the wire. A surface of the first portion facing the second portion is a curved surface. A surface of the second portion facing the first portion is a planar surface.

### BRIEF DESCRIPTION OF DRAWINGS

FIG. 1 is an explanatory view schematically illustrating a configuration of a guide wire according to a first embodiment.

FIG. 2 is an explanatory view schematically illustrating a configuration of the guide wire according to the first embodiment.

FIG. 3 is an explanatory view schematically illustrating a configuration of the guide wire according to the first embodiment.

FIG. 4 is an explanatory view schematically illustrating a configuration of the guide wire according to the first embodiment.

FIG. 5 is an explanatory view schematically illustrating a configuration of the guide wire according to the first embodiment.

FIG. 6 is a flowchart illustrating an example of a treatment method using the guide wire.

FIG. 7 is an explanatory view illustrating an example of the treatment method using the guide wire.

FIG. 8 is an explanatory view illustrating an example of the treatment method using the guide wire.

FIG. 9 is an explanatory view illustrating an example of the treatment method using the guide wire.

FIG. 10 is an explanatory view schematically illustrating a configuration of a guide wire according to a second embodiment.

FIG. 11 is an explanatory view schematically illustrating a configuration of the guide wire according to the second embodiment.

FIG. 12 is an explanatory view schematically illustrating a configuration of a guide wire according to a third embodiment.

FIG. 13 is an explanatory view schematically illustrating a configuration of the guide wire according to the third embodiment.

FIG. 14 is an explanatory view schematically illustrating a configuration of a guide wire according to a fourth embodiment.

FIG. 15 is an explanatory view schematically illustrating a configuration of the guide wire according to the fourth embodiment.

FIG. 16 is an explanatory view schematically illustrating a configuration of a guide wire according to a fifth embodiment.

FIG. 17 is an explanatory view schematically illustrating a configuration of the guide wire according to the fifth embodiment.

FIG. 18 is an explanatory view schematically illustrating a configuration of a guide wire according to a modification.

FIG. 19 is an explanatory view schematically illustrating a configuration of the guide wire according to the modification.

### DETAILED DESCRIPTION

### A. First Embodiment

### (Basic Configuration of Guide Wire 100)

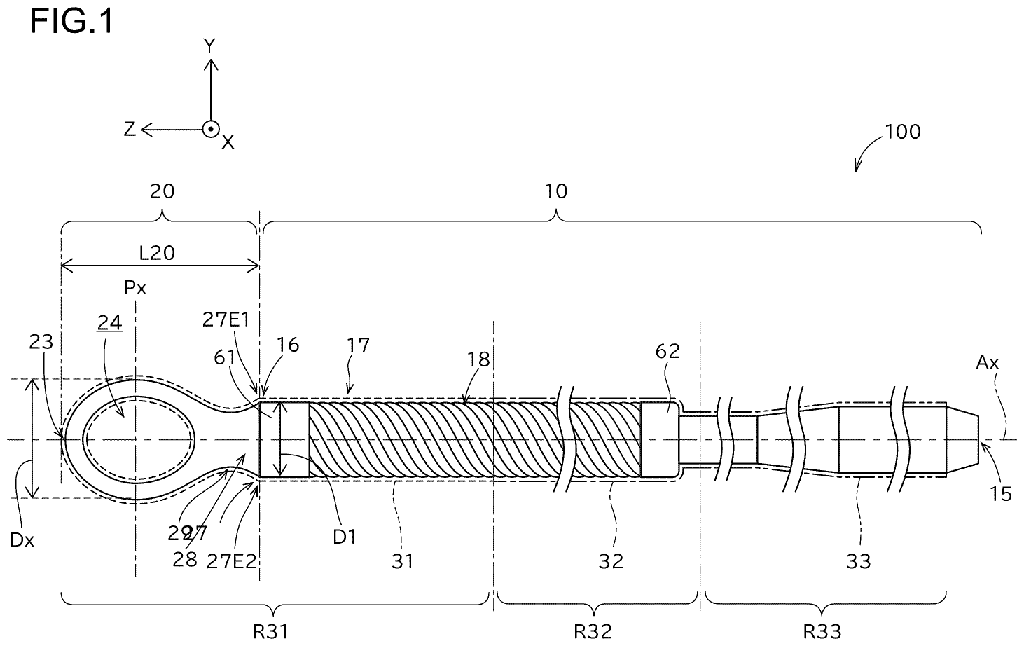

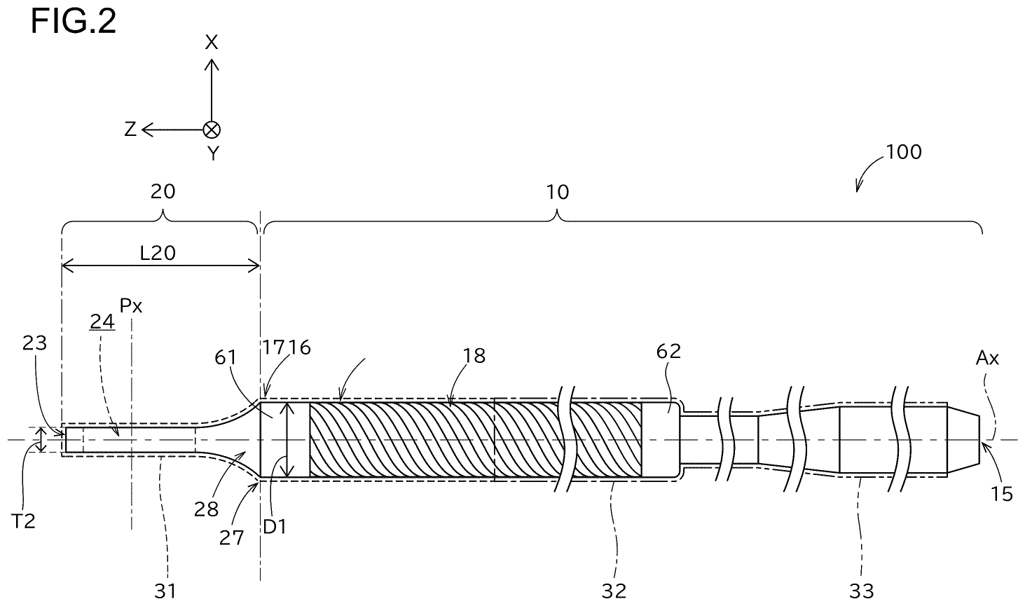

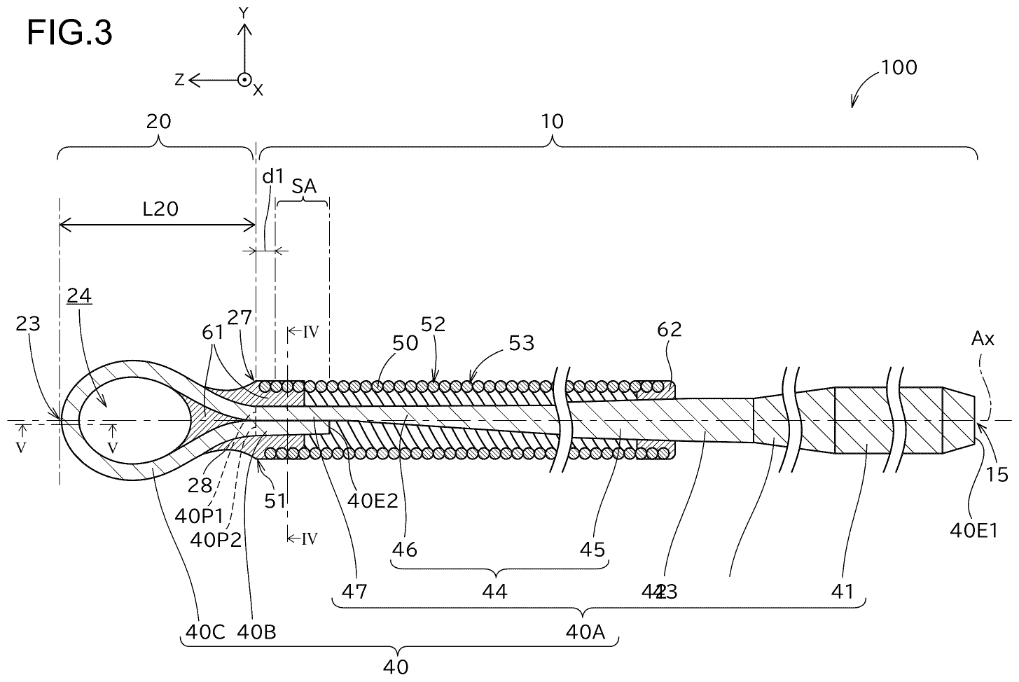

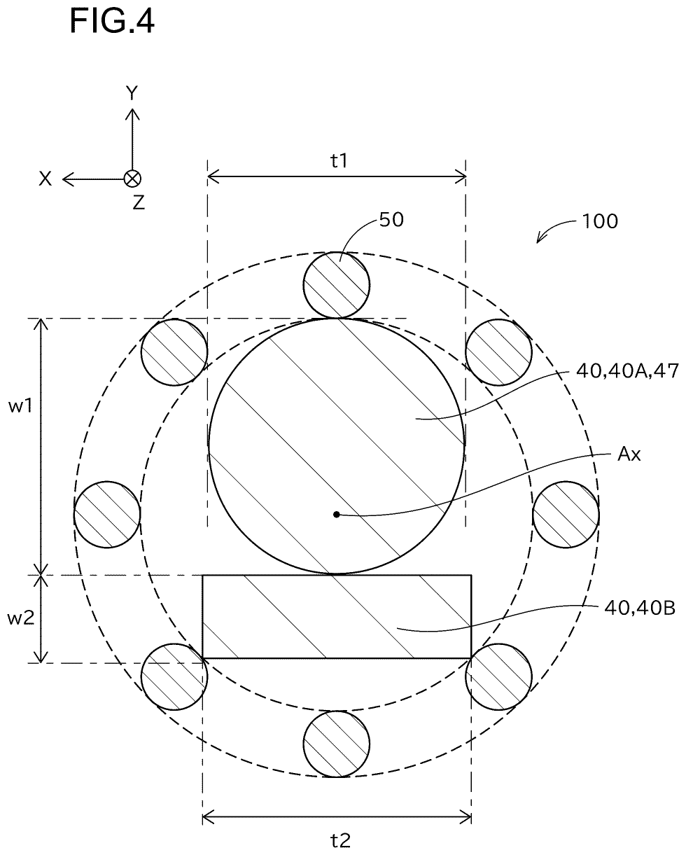

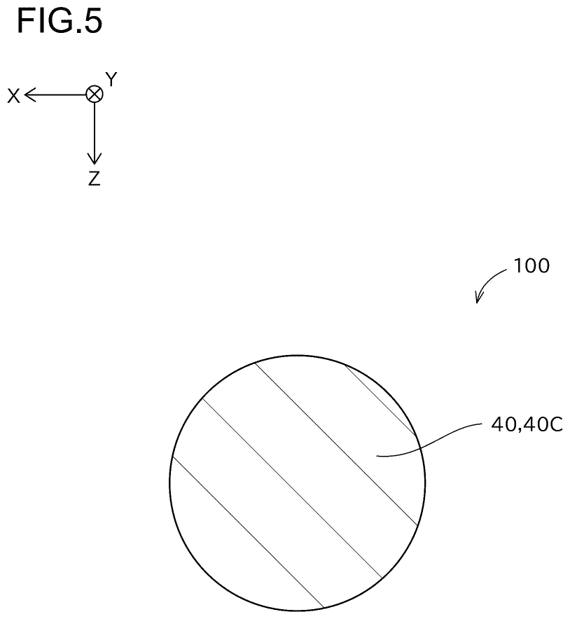

FIGS. 1 to 5 are explanatory views schematically illustrating a configuration of a guide wire 100 according to a first embodiment. In each drawing, XYZ axes orthogonal to each other for specifying a direction are illustrated. FIG. 1 illustrates an appearance of the guide wire 100 as viewed in an X-axis direction. FIG. 2 illustrates an appearance of the guide wire 100 as viewed in a Y-axis direction. FIG. 3 illustrates a YZ longitudinal section of the guide wire 100. FIG. 4 illustrates an XY transverse section of the guide wire 100 at a position IV-IV in FIG. 3. FIG. 5 illustrates an XZ cross section of the guide wire 100 at a position V-V in FIG. 3. In the guide wire 100, a Z-axis positive direction side is a distal end side (distal side) to be inserted into a body. In the guide wire 100, a Z-axis negative direction side is a proximal end side (proximal side) to be operated by a professional. In each drawing, a part of the guide wire 100 may be omitted. FIGS. 1 to 3 illustrate a state where the guide wire 100 is in a linear shape parallel to the Z-axis. The guide wire 100 is flexible enough to be curved. These points are also the same in the subsequent drawings. A Y-axis direction is an example of a first direction.

In the present specification, with respect to the guide wire 100 and each component thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. A transverse section of the guide wire 100 and each component thereof refers to a cross section that is orthogonal to a longitudinal direction. A longitudinal section of the guide wire 100 and each component thereof refers to a cross section that is parallel to a center axis in the longitudinal direction. With respect to the guide wire 100 and each component thereof, a direction orthogonal to the longitudinal direction is referred to as a “radial direction”. An outer diameter of the guide wire 100 and each component thereof refers to a width along the radial direction. In the present specification, with respect to the guide wire 100 and each component thereof, a length along the Y-axis direction may be particularly referred to as “width”, and a length along the X-axis direction may be particularly referred to as “thickness”.

The guide wire 100 is an elongated medical device to be inserted into a living body lumen such as a blood vessel. An entire length of the guide wire 100 is, for example, not less than 1000 mm and not more than 3000 mm. The guide wire 100 is an example of the medical device.

The guide wire 100 includes a main body part 10 and a leading portion 20.

The main body part 10 is an elongated portion extending along a center axis Ax. In the present embodiment, the center axis Ax of the main body part 10 coincides with the center axis of the guide wire 100. A proximal end 15 of the main body part 10 coincides with the proximal end of the guide wire 100. A spiral groove 18 is formed on an outer peripheral surface 17 of the distal end portion of the main body part 10.

The leading portion 20 is connected to a distal end 16 of the main body part 10. A proximal end 27 of the leading portion 20 is connected to the distal end 16 of the main body part 10. The leading portion 20 can be expressed as a guiding portion, a drilling portion, a fracturing portion, a peeling portion, an advancing portion, a peeler, a shaver, or the like. A distal end 23 of the leading portion 20 coincides with the distal end of the guide wire 100. The leading portion 20 has a loop shape surrounding a through-hole 24 extending in the X-axis direction. A surface of the leading portion 20 may have an edge or may not have an edge. The edge is a boundary (ridge line) between two surfaces. The leading portion 20 enters a lesion while rotating around the center axis Ax. The entry of the leading portion 20 into the lesion can be expressed as crossing/passing through the lesion, drilling the lesion, fracturing the lesion, peeling the lesion, pushing through the lesion, diving into the lesion, advancing into the lesion, and the like. A length L20 of the leading portion 20 along the center axis Ax is, for example, not less than 0.2 mm and not more than 2.0 mm. The length L20 of the leading portion 20 may be not less than 0.3 mm and not more than 1.5 mm, or may be not less than 0.4 mm and not more than 1.0 mm. When viewed in the X-axis direction, an end portion of the proximal end 27 of the leading portion 20 on a Y-axis positive direction side is referred to as a first end portion 27E1, and an end portion of the proximal end 27 of the leading portion 20 on a Y-axis negative direction side is referred to as a second end portion 27E2.

A maximum outer diameter Dx of the leading portion 20 is larger than a thickness T2 of the leading portion 20 at a maximum outer diameter position Px. That is, the leading portion 20 has a flat shape as a whole. The thickness T2 of the leading portion 20 at the maximum outer diameter position Px is smaller than a maximum outer diameter D1 of the distal end 16 of the main body part 10. The thickness T2 of the leading portion 20 at the maximum outer diameter position Px is, for example, not less than 0.02 mm and not more than 0.3 mm. The thickness T2 of the leading portion 20 at the maximum outer diameter position Px may be not less than 0.04 mm and not more than 0.2 mm, or may be not less than 0.06 mm and not more than 0.1 mm. The maximum outer diameter Dx of the leading portion 20 is, for example, not less than 1.2 times the thickness T2 of the leading portion 20 at the maximum outer diameter position Px. The maximum outer diameter Dx of the leading portion 20 may be not less than 1.5 times or not less than 1.8 times the thickness T2 of the leading portion 20 at the maximum outer diameter position Px.

The maximum outer diameter Dx of the leading portion 20 is measured as follows. A measurer observes the guide wire 100 from a side. The side is the Y-axis direction in the present embodiment. The measurer searches for an angle at which a portion on the near side and a portion on the far side of the leading portion 20 overlap each other and the portion on the far side cannot be seen. For example, when both of the following two conditions are satisfied, the measurer searches for an angle at which a portion between the second end portion 27E2 and the distal end 23 cannot be seen. The first condition is that a portion between the first end portion 27E1 and the distal end 23 is located on the near side. The second condition is that the portion between the second end portion 27E2 and the distal end 23 is located on the far side. The invisibility of the portion between the second end portion 27E2 and the distal end 23 is caused by an overlap between the portion between the first end portion 27E1 and the distal end 23 and the portion between the second end portion 27E2 and the distal end 23. Next, the measurer photographs the guide wire 100 by using a microscope along a viewpoint rotated by 90 degrees around the center axis Ax from the viewpoint at this time. The measurer sets a photographing magnification of the microscope to 200 times or more. The measurer measures the outer diameter of the leading portion 20 at three measurement positions where the leading portion 20 is considered to have the maximum outer diameter Dx on the captured image. To be specific, at each measurement position, the measurer draws a pair of straight lines in parallel to each other, which pass through a pair of end portions of the leading portion 20 in an outer diameter direction and are orthogonal to the outer diameter direction, and measures an interval between the pair of straight lines. The measurer adopts the maximum value of measurement results at the three measurement positions as the maximum outer diameter Dx of the leading portion 20.

### (Treatment Method Using Guide Wire 100)

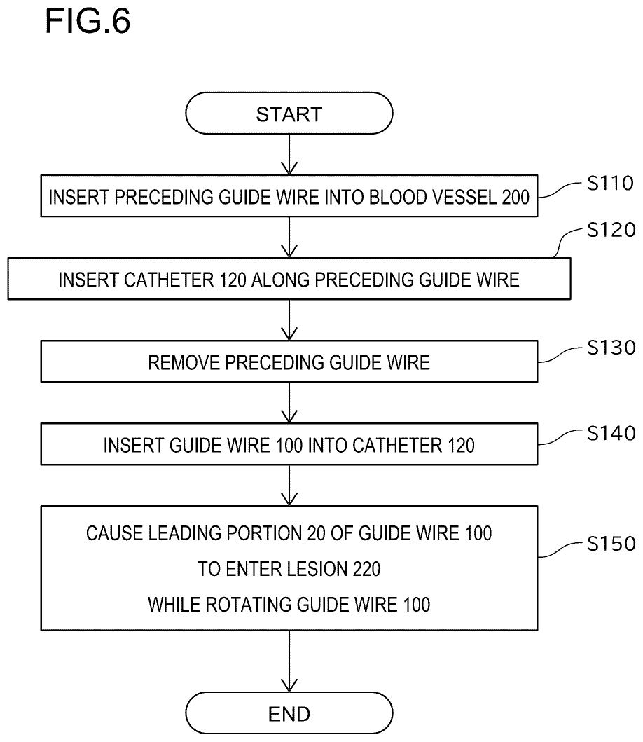

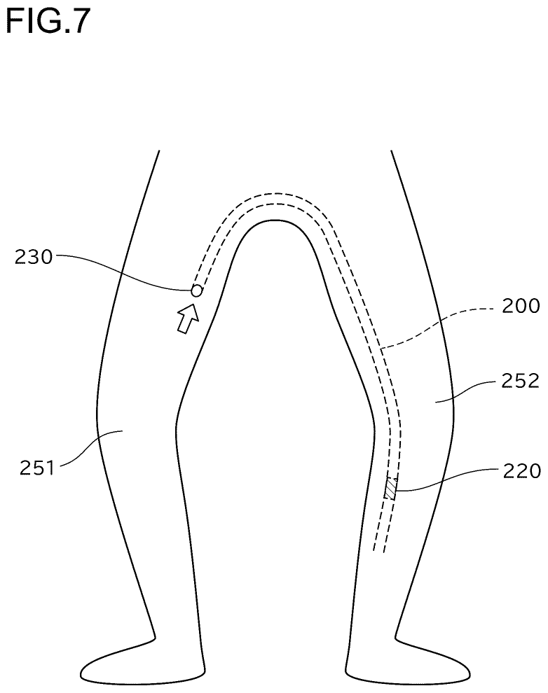

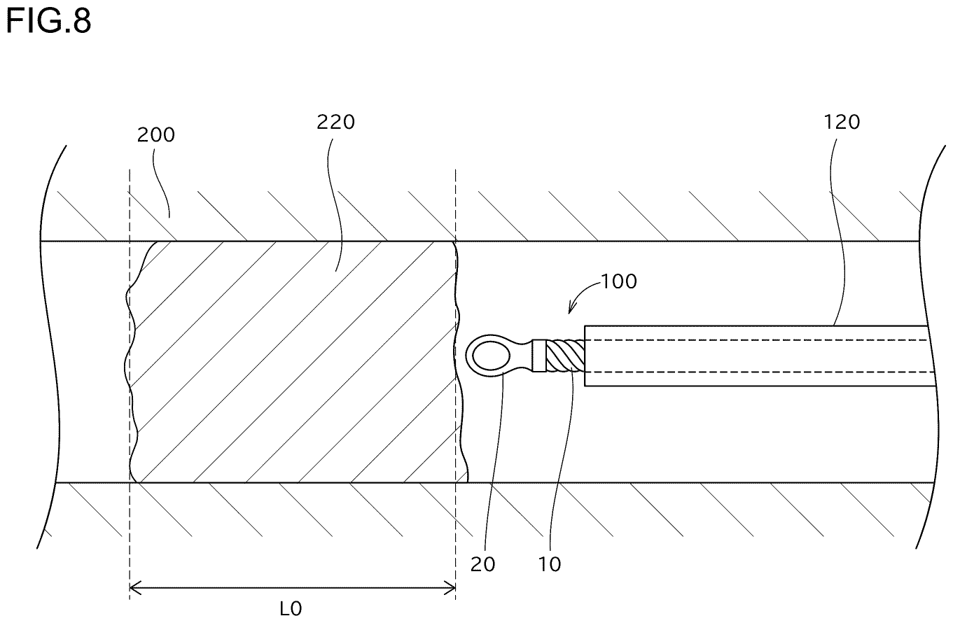

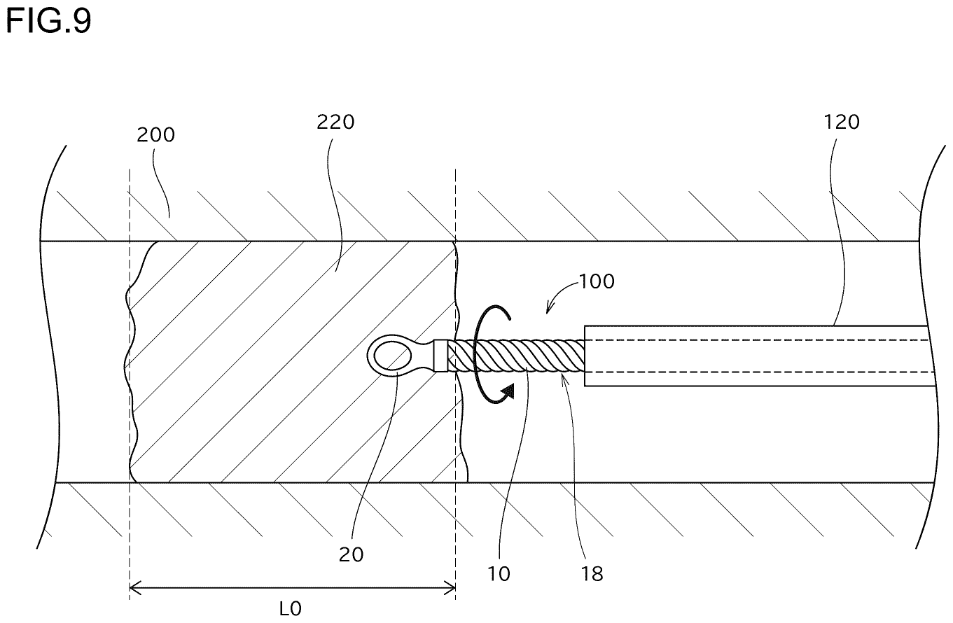

FIG. 6 is a flowchart illustrating an example of a treatment method using the guide wire 100. FIGS. 7 to 9 are explanatory views illustrating an example of the treatment method using the guide wire 100. As illustrated in FIGS. 8 and 9, in the treatment method using the guide wire 100, a professional causes the leading portion 20 of the guide wire 100 to enter a lesion 220 in a blood vessel 200. The lesion 220 is, for example, a highly calcified lesion. The lesion 220 is, for example, a chronic completely obstructive lesion. A length L0 of the lesion 220 along the extending direction of the blood vessel 200 is, for example, not less than 100 mm and not more than 500 mm. The length of the lesion 220 may be not less than 150 mm and not more than 450 mm, or not less than 200 mm and not more than 400 mm.

As illustrated in FIG. 7, the lesion 220 to be treated is located, for example, in the blood vessel 200 of the lower limb of a human. FIG. 7 illustrates a lesion 220 occurred in a below-knee region. In this treatment method, for example, a technology called crossover is used in which the blood vessel 200 is accessed from a groin of an opposite leg 251, which is the leg opposite to a target leg 252 in which the lesion 220 is located, and the lesion 220 in the target leg 252 is approached. A method of approaching the lesion 220 occurred in the below-knee region by inserting the guide wire 100 into the blood vessel 200 may be a method other than the crossover. The approach method may be an antegrade approach in which the guide wire 100 is inserted through the blood vessel 200 in the groin of the target leg 252 in which the lesion 220 is located, and the guide wire 100 is advanced along the flow of the blood stream. The approach method may be an antegrade approach in which the guide wire 100 is inserted through the blood vessel 200 in the arm and the guide wire 100 is advanced along the flow of the blood stream. The approach method may be a retrograde approach in which the guide wire 100 is inserted through either the blood vessel 200 in the ankle or the blood vessel 200 in the dorsum of the foot, of the target leg 252 in which the lesion 220 is located, and the guide wire 100 is advanced against the flow of the blood stream. The position at which the guide wire 100 is inserted into the blood vessel 200 is not limited to the above-described position. When the guide wire 100 is inserted through the blood vessel 200 in the arm, the professional can select the blood vessel 200 in the wrist as a position at which the guide wire 100 is inserted into the blood vessel 200. When the guide wire 100 is inserted through the blood vessel 200 in the leg, the professional can select a superficial femoral artery or a popliteal artery as the position at which the guide wire 100 is inserted into the blood vessel 200. The guide wire 100 is not limited to the treatment of the lesion 220 occurred in the below-knee region and can be used in the treatment of the lesion 220 occurred in another region such as an iliac artery.

First, the professional inserts a preceding guide wire into the blood vessel 200 (S110). Unlike the guide wire 100 of the present embodiment, the preceding guide wire is a known guide wire that does not have the leading portion 20. The preceding guide wire may be referred to as a workhorse guide wire, a first choice guide wire, or the like.

The professional inserts the preceding guide wire into the blood vessel 200 via a sheath (not illustrated) disposed at a puncture position 230 (see FIG. 7) of the opposite leg 251. The professional advances the preceding guide wire to a location immediately proximal to the lesion 220 in the blood vessel 200 of the target leg 252.

Next, the professional inserts a catheter 120 into the blood vessel 200 along the preceding guide wire (S120). The professional advances the catheter 120 (see FIG. 8) to a location immediately proximal to the lesion 220 in the blood vessel 200.

Next, the professional removes the preceding guide wire from the blood vessel 200 (S130). Thereafter, the professional inserts the guide wire 100 into the catheter 120 inserted into the blood vessel 200, with the leading portion 20 at the head (S140, FIG. 8). The professional advances the guide wire 100 to a location immediately proximal to the lesion 220 in the blood vessel 200. When advancing the guide wire 100, the professional may or may not rotate the guide wire 100 around the center axis Ax.

Next, the professional advances the guide wire 100 toward the distal end side while rotating the guide wire 100, thereby causing the leading portion 20 of the guide wire 100 to enter the lesion 220 (S150, FIG. 9). When the professional grips the proximal end portion of the guide wire 100 and rotates the guide wire 100 around the center axis Ax, the leading portion 20 located at the distal end portion of the guide wire 100 also rotates around the center axis Ax. The leading portion 20, which rotates within the lesion 220, drills the lesion 220 in a way that tears apart the lesion. In the present embodiment, in the step of advancing the guide wire 100 (S150), the professional advances the leading portion 20 until the leading portion 20 passes through the lesion 220. In a case where the leading portion 20 has an edge, when the leading portion 20 rotates around the center axis Ax in a state where the edge is in contact with the lesion 220, the edge scrapes the lesion 220. The leading portion 20 advances into a space formed by the edge scraping the lesion 220, thereby forming a through-hole in the lesion 220. As described above, the spiral groove 18 is formed on the outer peripheral surface 17 of the main body part 10. Due to the presence of the groove 18, the main body part 10 has a function of discharging small pieces of the lesion 220 generated by contact between the leading portion 20, which is rotating, and the lesion 220 from the distal end side toward the proximal end side of the main body part 10 (referred to as “lesion discharge function”). The step of advancing the guide wire 100 (S150) is performed in a state where no other medical devices have passed through the lesion 220.

After the leading portion 20 of the guide wire 100 has passed through the lesion 220, the professional advances a catheter (not illustrated) to the position of the lesion 220 along the guide wire 100. Thereafter, the professional removes the guide wire 100. When removing the guide wire 100, the professional may or may not rotate the guide wire 100 around the center axis Ax.

Thereafter, the professional inserts a guide wire for an adjunctive device (not illustrated) into the blood vessel 200 and advances the guide wire for an adjunctive device until the distal end of the guide wire for an adjunctive device passes through the lesion 220. The professional advances the adjunctive device to the position of the lesion 220 to lie along the guide wire for the adjunctive device. The adjunctive device may be, for example, an atherectomy device, a balloon catheter, a stent, and the like.

### (Detailed Configuration of Guide Wire 100)

As illustrated in FIG. 3, the guide wire 100 includes a wire 40 and a coil 50.

The coil 50 is a cylindrical member in which one or more wires are spirally wound. The main body part 10 includes the coil 50. An outer diameter of the coil 50 is, for example, not less than 0.1 mm and not more than 0.6 mm. The outer diameter of the coil 50 may be not less than 0.2 mm and not more than 0.5 mm, or may be not less than 0.3 mm and not more than 0.4 mm. The outer diameter of the coil 50 may be not less than 1.00 mm and not more than 2.00 mm, may be not less than 1.10 mm and not more than 1.65 mm, or may be not less than 1.20 mm and not more than 1.35 mm. In this embodiment, the outer diameter of the coil 50 is constant over the entire length of the coil 50. The coil 50 may have a tapered shape in which the outer diameter of the coil 50 gradually decreases from a proximal end toward a distal end, or may have a tapered shape in which the outer diameter of the coil 50 gradually decreases from the distal end toward the proximal end. A spiral groove 53 is formed on an outer peripheral surface 52 of the coil 50. In the present embodiment, the coil 50 is Z-wound, and a running direction of the groove 53 is also the Z-winding direction. Due to the presence of the spiral groove 53, the spiral groove 18 is formed on the outer peripheral surface 17 of the main body part 10. The distal end of the coil 50 coincides with the distal end 16 of the main body part 10. The coil 50 is an example of a tubular body.

The wire forming the coil 50 may be a single strand or a twisted wire formed by twisting a plurality of strands. In the present embodiment, the coil 50 is a multi-thread coil formed by winding a plurality of wires. In the present embodiment, each wire forming the coil 50 is a twisted wire.

As a material forming the coil 50, for example, a metal is used. To be more specific, for example, radiolucent materials such as stainless steels (SUS302, SUS304,SUS316, etc.), Ni—Ti alloys, piano wire, and radiopaque materials such as platinum, gold, tungsten, and any of their alloys may be used. The coil 50 may be wholly formed of the same material, or may be partially formed of materials different from each other.

The wire 40 is a linear member. The wire 40 includes a base portion 40A, a folded portion 40B, and a loop portion 40C. In the wire 40, the base portion 40A, the loop portion 40C, and the folded portion 40B are arranged in this order from a first end 40E1 toward a second end 40E2 of the wire 40. The base portion 40A and the loop portion 40C are continuous in a longitudinal direction of the wire 40. The loop portion 40C and the folded portion 40B are continuous in the longitudinal direction of the wire 40. The main body part 10 includes a base portion 40A and a folded portion 40B of the wire 40. The leading portion 20 includes a loop portion 40C of the wire 40. The base portion 40A is an example of a first portion. The folded portion 40B is an example of a second portion. The loop portion 40C is an example of a third portion.

The base portion 40A is a portion of the wire 40. A proximal end of the base portion 40A is located at a proximal end portion of the main body part 10. The proximal end side of the base portion 40A is a portion to be gripped by a professional. A distal end of the base portion 40A is located at the distal end portion of the main body part 10.

The base portion 40A includes a large diameter portion 41, a first tapered portion 42, an intermediate diameter portion 43, a second tapered portion 44, and a small diameter portion 47. In the base portion 40A, the large diameter portion 41, the first tapered portion 42, the intermediate diameter portion 43, the second tapered portion 44, and the small diameter portion 47 are arranged in this order from the proximal end side toward the distal end side of the guide wire 100.

The large diameter portion 41 is a rod-shaped portion having a substantially constant outer diameter. The outer diameter (maximum width) of the large diameter portion 41 is, for example, about not less than 0.2 mm and not more than 3.0 mm. The first tapered portion 42 is a portion whose diameter gradually decreases from a boundary with the large diameter portion 41 toward a boundary with the intermediate diameter portion 43. The intermediate diameter portion 43 is a rod-shaped portion having a substantially constant outer diameter smaller than the outer diameter of the large diameter portion 41. The second tapered portion 44 is a portion whose diameter gradually decreases from the boundary with the intermediate diameter portion 43 toward a boundary with the small diameter portion 47. In this embodiment, the second tapered portion 44 includes a proximal-end-side second tapered portion 45 and a distal-end-side second tapered portion 46 located on the distal end side with respect to the proximal-end-side second tapered portion 45. The small diameter portion 47 is a rod-shaped portion having a substantially constant outer diameter smaller than the outer diameter of the intermediate diameter portion 43.

In the present embodiment, rates of change (hereinafter, referred to as “gradient”) of the outer diameter along the longitudinal direction in the proximal-end-side second tapered portion 45 and the distal-end-side second tapered portion 46 are different from each other. For example, the gradient of the proximal-end-side second tapered portion 45 is steeper than the gradient of the distal-end-side second tapered portion 46. The gradient of the proximal-end-side second tapered portion 45 may be gentler than the gradient of the distal-end-side second tapered portion 46, or may be the same as the gradient of the distal-end-side second tapered portion 46. In the present embodiment, gradients of the first tapered portion 42 and the second tapered portion 44 are different from each other. For example, the gradient of the first tapered portion 42 is steeper than the gradient of the second tapered portion 44. The gradient of the first tapered portion 42 may be gentler than the gradient of the second tapered portion 44, or may be the same as the gradient of the second tapered portion 44.

The folded portion 40B is a portion of the wire 40 that is different from the base portion 40A. The folded portion 40B has a portion facing the base portion 40A in the Y-axis direction intersecting an axial direction of the main body part 10. In the present embodiment, the base portion 40A and the folded portion 40B are in contact with each other. The proximal end of the folded portion 40B is located on the distal end side with respect to the proximal end of the base portion 40A. In other words, the proximal end of the base portion 40A is located on the proximal end side with respect to the proximal end of the folded portion 40B. The distal end of the folded portion 40B is located at the distal end portion of the main body part 10.

The loop portion 40C is a portion of the wire 40 that is different from the base portion 40A and the folded portion 40B. The loop portion 40C is located between the base portion 40A and the folded portion 40B in the longitudinal direction of the wire 40.

The loop portion 40C has a loop shape when viewed in the X-axis direction. The loop portion 40C surrounds the through-hole 24 in the leading portion 20. The loop portion 40C extends from a boundary position 40P1, which is a boundary position with the base portion 40A, toward the distal end side, is folded back toward the proximal end side at the distal end 23 of the leading portion 20, and extends toward the proximal end side to a boundary position 40P2, which is a boundary position with the folded portion 40B. The boundary position 40P1 and the boundary position 40P2 are proximal ends of the loop portion 40C. The proximal end of the loop portion 40C is located at the proximal end portion of the leading portion 20. The distal end of the loop portion 40C is located at the distal end portion of the leading portion 20.

As a material forming the wire 40, for example, a metal is used. More specifically, for example, stainless steels (SUS302, SUS304, SUS316, etc.), Ni—Ti alloys, piano wires, and the like are used. The wire 40 may be wholly formed of the same material, or may be partially formed of materials different from each other.

The wire 40 is inserted into the hollow of the coil 50. The coil 50 covers at least a part of the distal end side of the base portion 40A and the folded portion 40B.

The coil 50 is joined to the wire 40 via a distal-end-side bonding material 61 formed at the distal end portion of the coil 50 and a proximal-end-side bonding material 62 formed at the proximal end portion of the coil 50. The base portion 40A and the folded portion 40B are connected to the coil 50 via the distal-end-side bonding material 61. The distal-end-side bonding material 61 protrudes from a distal end 51 of the coil 50 toward the distal end side. The loop portion 40C is connected to the coil 50 via a portion of the distal-end-side bonding material 61 protruding from the distal end 51 of the coil 50. The coil 50 may be joined to the wire 40 via a bonding material formed at another position. As a material for forming the distal-end-side bonding material 61 and the proximal-end-side bonding material 62, for example, a metal solder (Au—Sn alloy, Sn—Ag alloy, Sn—Pb alloy, Pb—Ag alloy, etc.), a brazing material (aluminum alloy braze, silver braze, gold braze, etc.), an adhesive (epoxy-based adhesive, etc.), or the like is used.

The leading portion 20 has a reinforcing portion 28 located at a connection portion with the main body part 10. The connection portion of the leading portion 20 with the main body part 10 is reinforced by the reinforcing portion 28. In the present embodiment, the reinforcing portion 28 is formed of a portion of the distal-end-side bonding material 61 that protrudes from the distal end 51 of the coil 50 toward the distal end side. The reinforcing portion 28 may be formed by a welded portion between the leading portion 20 and the main body part 10. A constriction 29 is formed on an outer peripheral surface of the reinforcing portion 28. That is, a width of the reinforcing portion 28 in a direction orthogonal to the axis Ax decreases toward a distal end of the reinforcing portion 28 in a part (for example, a proximal end portion) of the reinforcing portion 28, and a width of the reinforcing portion 28 in the direction orthogonal to the axis Ax increases toward the distal end of the reinforcing portion 28 in another part (for example, a distal end portion) of the reinforcing portion 28. Therefore, the outer peripheral surface of the reinforcing portion 28 has a curved surface that is recessed inward in a radial direction of the reinforcing portion 28.

As illustrated in FIG. 1, the guide wire 100 includes a first coating 31 and a second coating 32. The first coating 31 covers at least a part of the leading portion 20. The first coating 31 may cover a part of the main body part 10. In the present embodiment, the first coating 31 covers a region R31 formed by the entire leading portion 20 and the distal end portion of the main body part 10. The second coating 32 covers at least a part of the main body part 10. In the present embodiment, the second coating 32 covers a region R32 which is an intermediate portion of the main body part 10 excluding the distal end portion and the proximal end portion. The region R32 is located on a proximal end side of the region R31. A slidability of the first coating 31 is different from a slidability of the second coating 32. For example, the slidability of the first coating 31 is lower than the slidability of the second coating 32. The first coating 31 is, for example, hydrophobic and is formed of silicone. The second coating 32 is, for example, hydrophilic, and is formed of polyvinylpyrrolidone, polyacrylic acid, polyacrylamide, polyvinyl alcohol, a maleic anhydride copolymer, hyaluronic acid, or the like.

The guide wire 100 further includes a third coating 33. The third coating 33 covers at least a part of the main body part 10. In the present embodiment, the third coating 33 covers a region R33, which is the proximal end portion of the main body part 10. The region R33 is located on the proximal end side of the region R32. The third coating 33 is formed of, for example, PTFE.

As illustrated in FIG. 4, a shape of the transverse section of the base portion 40A and a shape of the transverse section of the folded portion 40B are different from each other. A surface of the base portion 40A facing the folded portion 40B is a curved surface. The “curved surface” in this specification includes not only a geometrically ideal curved surface but also shapes that deviate from a curved surface due to unavoidable circumstances such as manufacturing errors. In the present embodiment, the transverse section of the base portion 40A is circular. More specifically, the transverse section of the base portion 40A is a perfect circle. The transverse section of the base portion 40A may have a circular shape other than a perfect circle, such as an ellipse. A surface of the folded portion 40B facing the base portion 40A is a planar surface. The “planar surface” in this specification includes not only a geometrically ideal planar surface but also shapes that deviate from a planar surface due to unavoidable circumstances such as manufacturing errors. In the present embodiment, the transverse section of the folded portion 40B is rectangular. More specifically, the transverse section of the folded portion 40B is an oblong. The transverse section of the folded portion 40B may have a rectangular shape other than an oblong, such as a square, a trapezoid, or a parallelogram.

In the present specification, a region in which the surface of the base portion 40A facing the folded portion 40B is a curved surface and the surface of the folded portion 40B facing the base portion 40A is a planar surface is referred to as a specific region SA. A distance d1 from the distal end 16 of the main body part 10 to a distal end of the specific region SA is, for example, 2 mm or less. The distance d1 may be 1.6 mm or less, or may be 1.2 mm or less.

In the present embodiment, as illustrated in FIG. 4, in the Y-axis direction in which the base portion 40A and the folded portion 40B face each other, when a width of the base portion 40A is defined as width w1 and a width of the folded portion 40B is defined as width w2, the relation w1>w2 holds. That is, the width w1 in the Y-axis direction of the distal end portion of the base portion 40A is smaller than the width w2 in the Y-axis direction of the distal end portion of the folded portion 40B. A ratio w1/w2 between the width w1 and the width w2 is, for example, not less than 1.1 and not more than 2.0. w1/w2 may be not less than 1.2 and not more than 1.8, or may be not less than 1.25 and not more than 1.5. The width w1 and the width w2 may be such that w1<w2 or w1=w2. In the present embodiment, in the X-axis direction, when a thickness of the base portion 40A is defined as thickness t1 and a thickness of the folded portion 40B is defined as thickness t2, the relation t1<t2 holds. The thickness t1 and the thickness t2 may be such that t1>t2 or t1=t2.

As illustrated in FIG. 5, in this embodiment, the transverse section of the loop portion 40C is circular. More specifically, the transverse section of the loop portion 40C is a perfect circle. In other words, a shape of the transverse section of the base portion 40A and a shape of the transverse section of the loop portion 40C are similar to each other. The transverse section of the loop portion 40C may have a circular shape other than a perfect circle, such as an ellipse.

### (Method of Manufacturing Guide Wire 100)

The guide wire 100 of the present embodiment can be manufactured, for example, by the following manufacturing method. First, a worker prepares the wire 40. The worker performs, for example, press working, polishing, or the like on the wire 40 to make at least part of the surface of the portion corresponding to the folded portion 40B planar. That is, the worker performs, for example, press working, polishing, or the like to make the transverse section of the portion corresponding to the folded portion 40B rectangular. Next, the worker inserts the wire 40 into a hollow portion of the coil 50. At this time, the worker arranges the wire 40 in the hollow portion of the coil 50 so that part of the wire 40 on the distal end side protrudes from the distal end of the coil 50. Next, the worker bends the portion of the wire 40 protruding from the distal end of the coil 50 into a loop shape. As a result, portions corresponding to the base portion 40A, the folded portion 40B, and the loop portion 40C of the wire 40 are formed. Next, the worker inserts the portion of the wire 40 that corresponds to the folded portion 40B into the hollow portion of the coil 50.

Next, the worker joins the wire 40 and the coil 50. Specifically, to join the wire 40 to the coil 50, the worker forms the distal-end-side bonding material 61 and the proximal-end-side bonding material 62 with a bonding material such as a solder material. The bonding material is supplied, for example, between the strands of the coil 50 to the inside of the coil 50. The reinforcing portion 28 is formed by the distal-end-side bonding material 61 protruding from the distal end of the coil 50 toward the distal end side. By the joining step, the guide wire 100 including the leading portion 20 having the reinforcing portion 28 and the main body part 10 is manufactured.

### (Effect of Present Embodiment)

As described above, the guide wire 100 of the present embodiment includes the elongated main body part 10 and the leading portion 20 that is connected to the distal end 16 of the main body part 10 and enters the lesion 220. The main body part 10 includes a base portion 40A, which is a portion of the wire 40, and a folded portion 40B, which is a portion of the wire 40 different from the base portion 40A and has a portion facing the base portion 40A in the Y-axis direction intersecting the axial direction of the main body part 10. The leading portion 20 includes a loop portion 40C that is a portion of the wire 40 different from the base portion 40A and the folded portion 40B and is located between the base portion 40A and the folded portion 40B of the wire 40. A surface of the base portion 40A facing the folded portion 40B is a curved surface. A surface of the folded portion 40B facing the base portion 40A is a planar surface. According to the guide wire 100 of the present embodiment, the position of the base portion 40A and the position of the folded portion 40B are easily determined. When the folded portion 40B is formed so as to overlap the base portion 40A by folding back the wire 40, since one of the base portion 40A and the folded portion 40B is a planar surface, the base portion 40A and the folded portion 40B are suppressed from being displaced from each other in a direction (X-axis direction) orthogonal to the axial direction of the main body part 10. Accordingly, the base portion 40A and the folded portion 40B are suppressed from deviating and intersecting with each other. In other words, since the base portion 40A and the folded portion 40B are provided at the same position in the direction (X-axis direction) orthogonal to the axial direction of the main body part 10, the base portion 40A and the folded portion 40B extend in parallel to an axial direction (Z-axis direction) of the main body part 10.

In the guide wire 100 of the present embodiment, the transverse section of the base portion 40A is circular. According to the guide wire 100 of the present embodiment, the position of the base portion 40A and the position of the folded portion 40B are easily determined.

In the guide wire 100 of the present embodiment, the transverse section of the folded portion 40B is rectangular. According to the guide wire 100 of the present embodiment, the position of the base portion 40A and the position of the folded portion 40B are easily determined.

In the guide wire 100 of the present embodiment, the transverse section of the loop portion 40C is circular. According to the guide wire 100 of the present embodiment, the position of the base portion 40A and the position of the folded portion 40B are easily determined.

In the guide wire 100 of the present embodiment, the shape of the transverse section of the base portion 40A and the shape of the transverse section of the loop portion 40C are similar to each other. According to the guide wire 100 of the present embodiment, the position of the base portion 40A and the position of the folded portion 40B are easily determined.

In the guide wire 100 of the present embodiment, the proximal end of the base portion 40A is located at the proximal end portion of the main body part 10. According to the guide wire 100 of the present embodiment, the position of the base portion 40A and the position of the folded portion 40B are easily determined.

In the guide wire 100 of the present embodiment, the width in the Y-axis direction at the distal end portion of the base portion 40A is greater than the width in the Y-axis direction at the distal end portion of the folded portion 40B. According to the guide wire 100 of the present embodiment, since the width of the base portion 40A located at the proximal end portion of the main body part 10 is greater than the width of the folded portion 40B, torque transmissibility of the guide wire 100 is improved.

In the guide wire 100 of the present embodiment, the main body part 10 further includes a coil 50 that covers the base portion 40A and the folded portion 40B, and the base portion 40A and the folded portion 40B are joined to the coil 50. According to the guide wire 100 of the present embodiment, the position of the base portion 40A and the position of the folded portion 40B are easily determined.

In the guide wire 100 of the present embodiment, the proximal end of the base portion 40A is located on the proximal end side with respect to the proximal end of the folded portion 40B. According to the guide wire 100 of the present embodiment, the position of the base portion 40A and the position of the folded portion 40B are easily determined.

According to the guide wire 100 of the present embodiment, since the transverse section of the base portion 40A located at the proximal end portion of the main body part 10 is circular, the wire 40 is less likely to be twisted when the guide wire 100 is rotated, and the torque transmissibility of the guide wire 100 is improved.

### B. Second Embodiment

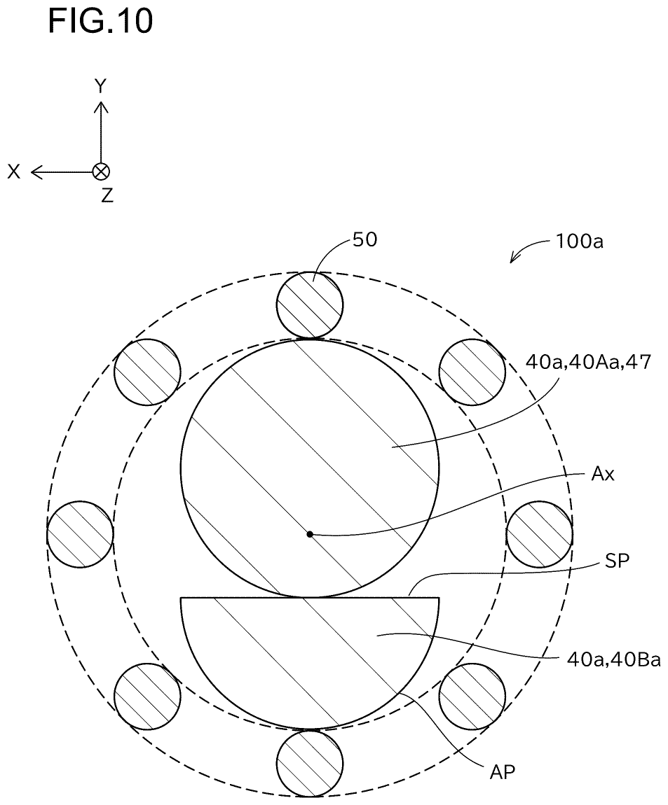

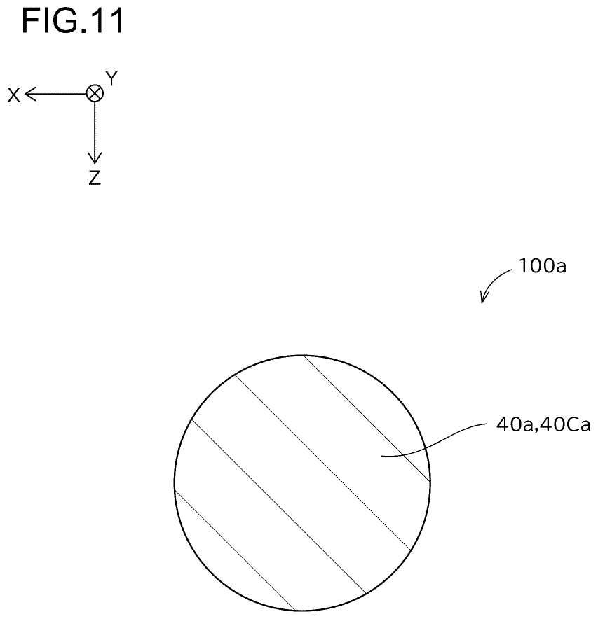

FIGS. 10 and 11 are explanatory views schematically illustrating a configuration of a guide wire 100a according to a second embodiment. FIG. 10 illustrates an XY transverse section of the guide wire 100a at the same location as the XY transverse section of guide wire 100 illustrated in FIG. 4. FIG. 11 illustrates an XZ cross section of the guide wire 100a at the same location as the XZ cross section of the guide wire 100 illustrated in FIG. 5. Hereinafter, components of the guide wire 100a according to the second embodiment, which are the same as the components of the guide wire 100 according to the first embodiment, are denoted with the same reference signs, thereby omitting the description thereof as appropriate.

In the guide wire 100a of the second embodiment, a shape of a transverse section of a wire 40a is different from the shape of the transverse section of the wire 40 in the guide wire 100 of the first embodiment. In detail, as illustrated in FIG. 10, a surface of a base portion 40Aa facing a folded portion 40Ba is a curved surface. In the present embodiment, a transverse section of the base portion 40Aa is circular. To be more specific, the transverse section of the base portion 40Aa is a perfect circle, similarly to the base portion 40A of the first embodiment. The transverse section of the base portion 40Aa may have a circular shape other than a perfect circle, such as an ellipse. A surface of the folded portion 40Ba facing the base portion 40Aa is a planar surface. In the present embodiment, an outer edge of the transverse section of the folded portion 40Ba includes an arc portion AP and a straight portion SP. The straight portion SP is located at a position facing the base portion 40Aa on the outer edge of the folded portion 40Ba. The arc portion AP is connected to both end portions of the straight portion SP. That is, in the present embodiment, the transverse section of the folded portion 40Ba is partially circular. More specifically, the transverse section of the folded portion 40Ba is a semicircle. The transverse section of the folded portion 40Ba may have a partial circular shape other than a semicircle, such as a circular segment or an arcuate shape. In the present specification, the partial circular shape is one of the two regions formed when a circle is divided by a chord. The semicircle is one of the two equal regions formed when a circle is divided by a chord that passes through the center of the circle. The circular segment is the larger of the two regions formed when a circle is divided by a chord that does not pass through the center of the circle. The arcuate shape is the smaller of the two regions formed when a circle is divided by a chord that does not pass through the center of the circle. The base portion 40Aa is an example of a first portion. The folded portion 40Ba is an example of a second portion.

As illustrated in FIG. 11, the transverse section of a loop portion 40Ca is circular. More specifically, the transverse section of the loop portion 40Ca is a perfect circle. The transverse section of the loop portion 40Ca may have a circular shape other than a perfect circle, such as an ellipse. The loop portion 40Ca is an example of a third portion.

As described above, in the guide wire 100a of the present embodiment, an outer edge of the transverse section of the folded portion 40Ba includes an arc portion AP and a straight portion SP, and the straight portion SP faces the base portion 40Aa. According to the guide wire 100a of the present embodiment, for example, it is possible to increase an area of the transverse section of the folded portion 40Ba as compared with a guide wire in which the transverse section of a folded portion is circular and a width of the folded portion in a first direction is equal to that of the folded portion 40Ba of the guide wire 100a. That is, according to the guide wire 100a of the present embodiment, it is easy to increase the size of the folded portion 40Ba.

In the guide wire 100a of the present embodiment, the transverse section of the folded portion 40Ba is partially circular. According to the guide wire 100a of the present embodiment, for example, it is possible to increase an area of the transverse section of the folded portion 40Ba as compared with a guide wire in which the transverse section of a folded portion is circular and a width of the folded portion in a first direction is equal to that of the folded portion 40Ba of the guide wire 100a. That is, according to the guide wire 100a of the present embodiment, it is easy to increase the size of the folded portion 40Ba.

### C. Third Embodiment

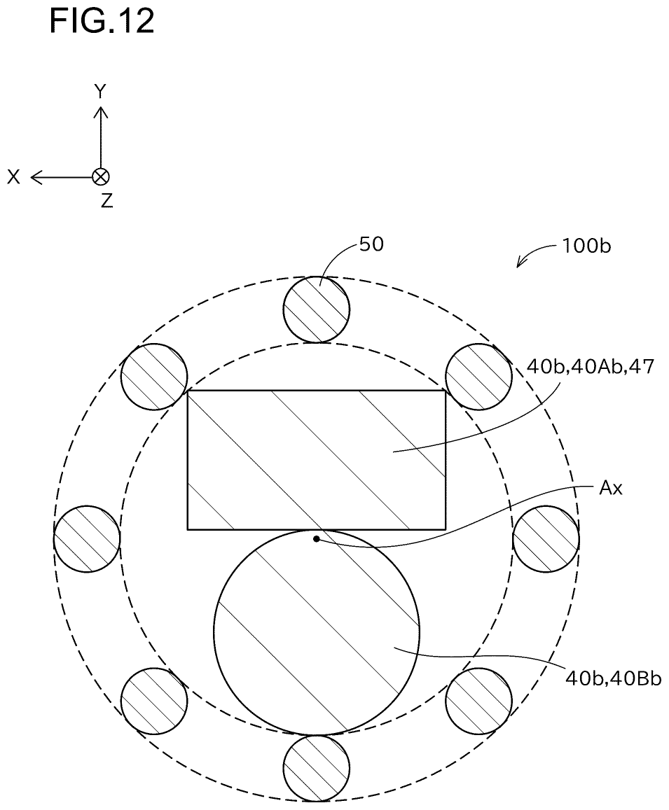

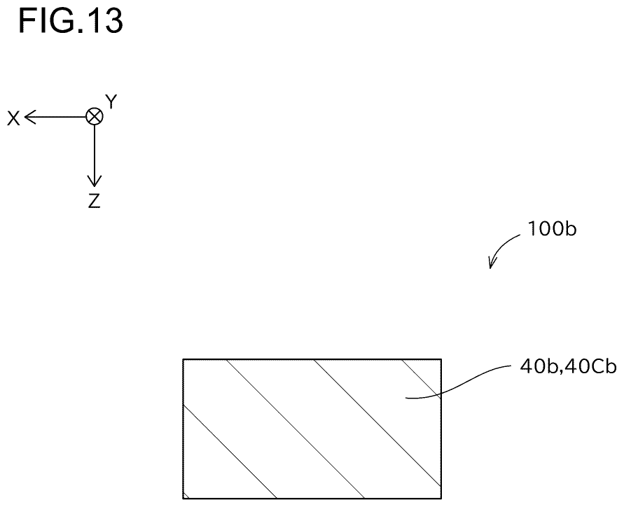

FIGS. 12 and 13 are explanatory views schematically illustrating a configuration of a guide wire 100b according to a third embodiment. FIG. 12 illustrates an XY transverse section of the guide wire 100b at the same location as the XY transverse section of the guide wire 100 illustrated in FIG. 4. FIG. 13 illustrates an XZ cross section of the guide wire 100b at the same location as the XZ cross section of the guide wire 100 illustrated in FIG. 5. Hereinafter, components of the guide wire 100b according to the third embodiment, which are the same as the components of the guide wire 100 according to the first embodiment, are denoted with the same reference signs, thereby omitting the description thereof as appropriate.

In the guide wire 100b of the third embodiment, a shape of a transverse section of a wire 40b is different from the shape of the transverse section of the wire 40 in the guide wire 100 of the first embodiment. In detail, as illustrated in FIG. 12, a surface of the base portion 40Ab facing a folded portion 40Bb is a planar surface. In the present embodiment, a transverse section of the base portion 40Ab is rectangular. More specifically, the transverse section of the base portion 40Ab is oblong. The transverse section of the base portion 40Ab may have a rectangular shape other than an oblong, such as a square, a trapezoid, or a parallelogram. A surface of the folded portion 40Bb facing the base portion 40Ab is a curved surface. In the present embodiment, the transverse section of the folded portion 40Bb is circular. More specifically, the transverse section of the folded portion 40Bb is a perfect circle. The transverse section of the folded portion 40Bb may have a circular shape other than a perfect circle, such as an ellipse. The folded portion 40Bb is an example of a first portion. The base portion 40Ab is an example of a second portion.

As illustrated in FIG. 13, the transverse section of the loop portion 40Cb is rectangular. More specifically, the transverse section of the loop portion 40Cb is oblong. The transverse section of the loop portion 40Cb may have a rectangular shape other than an oblong, such as a square or a trapezoid. The loop portion 40Cb is an example of a third portion.

As described above, in the guide wire 100b of the present embodiment, the transverse section of the loop portion 40Cb is rectangular. According to the guide wire 100b of the present embodiment, since the transverse section of the loop portion 40Cb is rectangular, flexibility of the loop portion 40Cb is improved as compared to, for example, a guide wire in which the transverse section of a loop portion is circular and an area of the transverse section of the loop portion is the same as that of the guide wire 100b.

### D. Fourth Embodiment

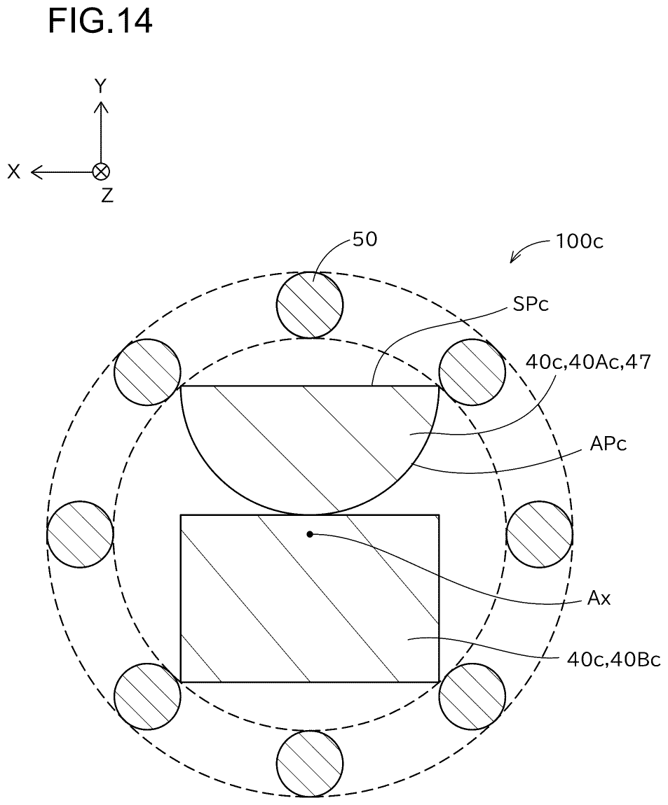

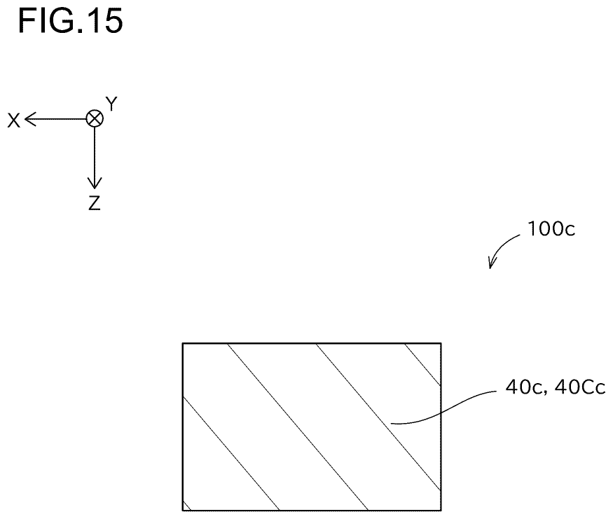

FIGS. 14 and 15 are explanatory views schematically illustrating a configuration of a guide wire 100c according to a fourth embodiment. FIG. 14 illustrates an XY transverse section of the guide wire 100c at the same location as the XY transverse section of the guide wire 100 illustrated in FIG. 4. FIG. 15 illustrates an XZ cross section of the guide wire 100c at the same location as the XZ cross section of the guide wire 100 illustrated in FIG. 5. Hereinafter, components of the guide wire 100c according to the fourth embodiment, which are the same as the components of the guide wire 100 according to the first embodiment, are denoted with the same reference signs, thereby omitting the description thereof as appropriate.

In the guide wire 100c of the fourth embodiment, a shape of a transverse section of a wire 40c is different from the shape of the transverse section of the wire 40 in the guide wire 100 of the first embodiment. In detail, as illustrated in FIG. 14, a surface of a base portion 40Ac facing a folded portion 40Bc is a curved surface. In the present embodiment, an outer edge of a transverse section of the base portion 40Ac includes an arc portion APc and a straight portion SPc. The arc portion APc is located at a position facing the folded portion 40Bc on the outer edge of the base portion 40Ac. The straight portion SPc is connected to both end portions of the arc portion APc. That is, in the present embodiment, the transverse section of the base portion 40Ac is partially circular. More specifically, the transverse section of the base portion 40Ac is a semicircle. The transverse section of the base portion 40Ac may have a partial circular shape other than a semicircle, such as a circular segment or an arcuate shape. A surface of the folded portion 40Bc facing the base portion 40Ac is a planar surface. In the present embodiment, a transverse section of the folded portion 40Bc is rectangular. More specifically, the transverse section of the folded portion 40Bc is oblong. The transverse section of the folded portion 40Bc may have a rectangular shape other than an oblong, such as a square or a trapezoid. The base portion 40Ac is an example of a first portion. The folded portion 40Bc is an example of a second portion.

As illustrated in FIG. 15, the transverse section of the loop portion 40Cc is rectangular. More specifically, the transverse section of the loop portion 40Cc is oblong. That is, the shape of the transverse section of the folded portion 40Bc and the shape of the transverse section of the loop portion 40Cc are similar to each other. The transverse section of the loop portion 40Cc may have a rectangular shape other than an oblong, such as a square or a trapezoid. The loop portion 40Cc is an example of a third portion.

As described above, in the guide wire 100c of the present embodiment, the outer edge of the transverse section of the base portion 40Ac includes an arc portion APc and a straight portion SPc, and the arc portion APc faces the folded portion 40Bc. According to the guide wire 100c of the present embodiment, the position of the base portion 40Ac and the position of the folded portion 40Bc are easily determined.

In the guide wire 100c of the present embodiment, the transverse section of the base portion 40Ac is partially circular. According to the guide wire 100c of the present embodiment, the position of the base portion 40Ac and the position of the folded portion 40Bc are easily determined.

In the guide wire 100c of the present embodiment, the shape of the transverse section of the folded portion 40Bc and the shape of the transverse section of the loop portion 40Cc are similar to each other. According to the guide wire 100c of the present embodiment, the position of the base portion 40Ac and the position of the folded portion 40Bc are easily determined.

### E. Fifth Embodiment

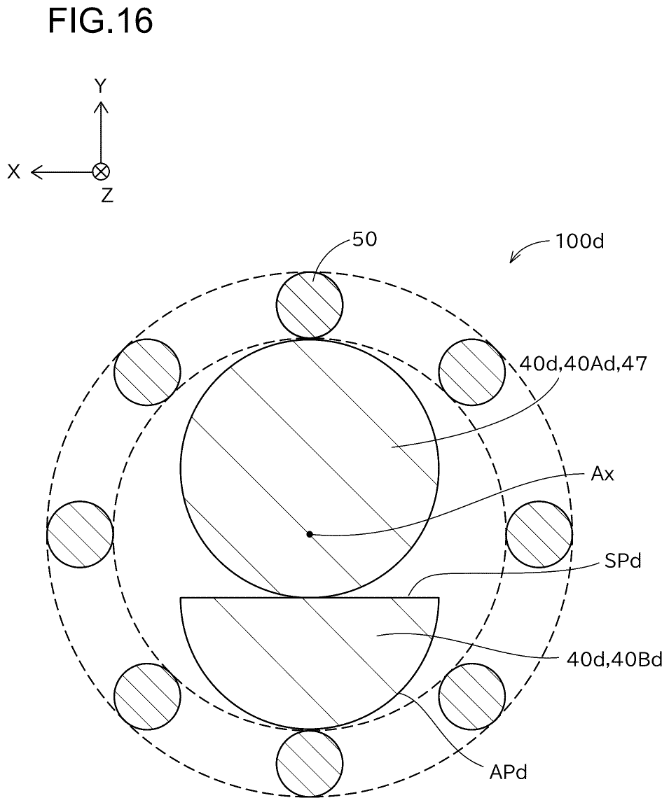

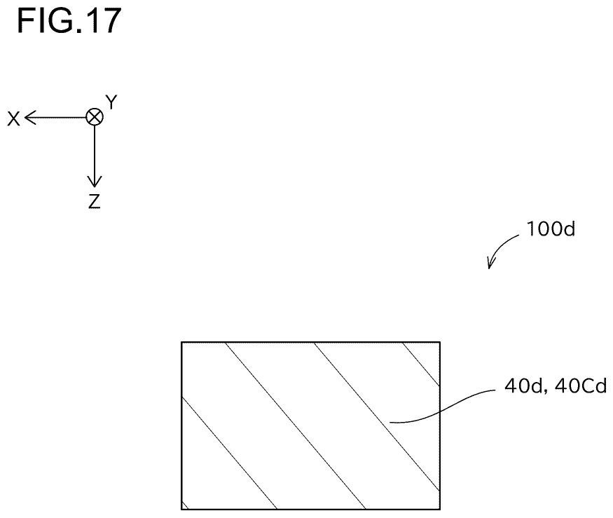

FIGS. 16 and 17 are explanatory views schematically illustrating a configuration of a guide wire 100d according to a fifth embodiment. FIG. 16 illustrates an XY transverse section of the guide wire 100d at the same location as the XY transverse section of the guide wire 100 illustrated in FIG. 4. FIG. 17 illustrates an XZ cross section of the guide wire 100d at the same location as the XZ cross section of the guide wire 100 illustrated in FIG. 5. Hereinafter, components of the guide wire 100d according to the fifth embodiment, which are the same as the components of the guide wire 100 according to the first embodiment, are denoted with the same reference signs, thereby omitting the description thereof as appropriate.

In the guide wire 100d of the fifth embodiment, a shape of a transverse section of a wire 40d is different from the shape of the transverse section of the wire 40 in the guide wire 100 of the first embodiment. In detail, as illustrated in FIG. 16, a surface of a base portion 40Ad facing a folded portion 40Bd is a curved surface. In the present embodiment, the transverse section of the base portion 40Ad is circular. To be more specific, the transverse section of the base portion 40Ad is a perfect circle, similarly to the base portion 40A of the first embodiment. The transverse section of the base portion 40Ad may have a circular shape other than a perfect circle, such as an ellipse. A surface of the folded portion 40Bd facing the base portion 40Ad is a planar surface. In the present embodiment, an outer edge of a transverse section of the folded portion 40Bd includes an arc portion APd and a straight portion SPd. The straight portion SPd is located at a position facing the base portion 40Ad on the outer edge of the folded portion 40Bd. The arc portion APd is connected to both end portions of the straight portion SPd. That is, in the present embodiment, the transverse section of the folded portion 40Bd is partially circular. More specifically, the transverse section of the folded portion 40Bd is a semicircle. The transverse section of the folded portion 40Bd may have a partial circular shape other than a semicircle, such as a circular segment or an arcuate shape. The base portion 40Ad is an example of a first portion. The folded portion 40Bd is an example of a second portion.

As illustrated in FIG. 17, the transverse section of the loop portion 40Cd is rectangular. More specifically, the transverse section of the loop portion 40Cd is oblong. That is, the shape of the transverse section of the base portion 40Ad, the shape of the transverse section of the folded portion 40Bd, and the shape of the transverse section of the loop portion 40Cd are not similar to one another. The transverse section of the loop portion 40Cd may be a rectangular shape other than an oblong, such as a square or a trapezoid. The loop portion 40Cd is an example of a third portion.

As described above, in the guide wire 100d of the present embodiment, the shape of the transverse section of the base portion 40Ad, the shape of the transverse section of the folded portion 40Bd, and the shape of the transverse section of the loop portion 40Cd are not similar to one another. According to the guide wire 100d of the present embodiment, the position of the base portion 40Ad and the position of the folded portion 40Bd are easily determined.

### F. Modifications

The technology disclosed in this specification is not limited to the embodiments described above but can be modified in various forms without departing from the gist of this specification, and, for example, the following modifications can be made.

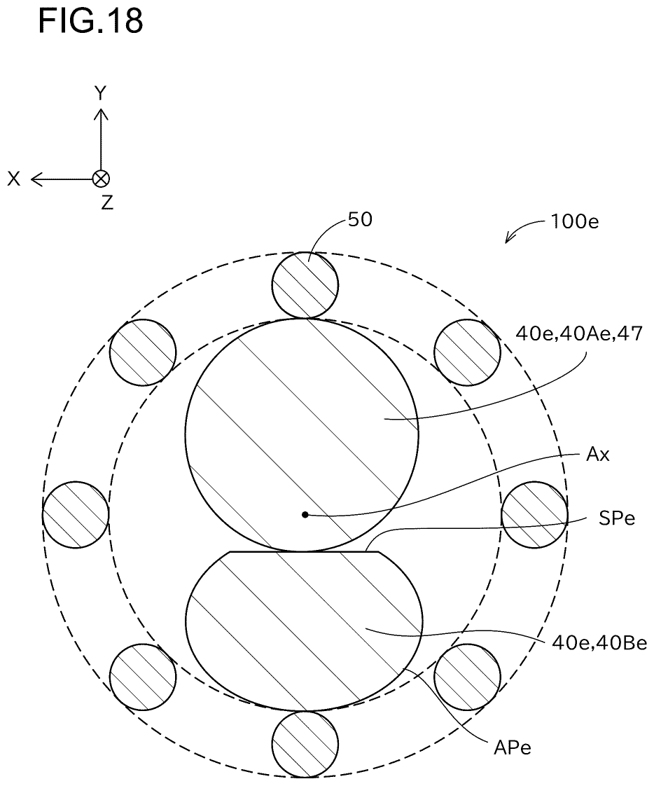

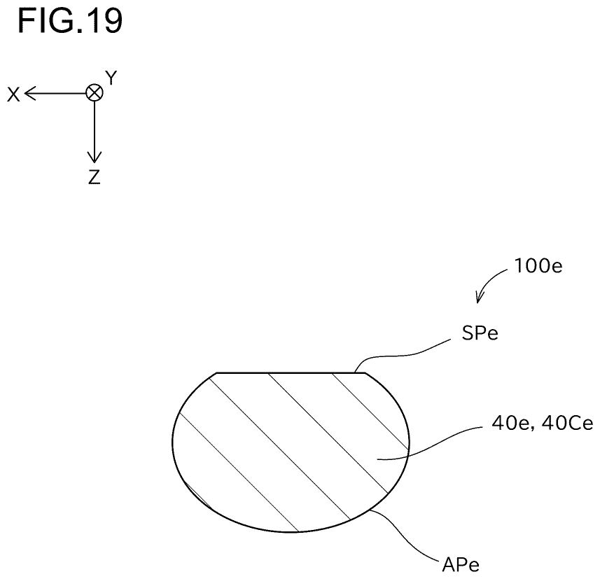

FIGS. 18 and 19 are explanatory views schematically illustrating a configuration of a guide wire 100e according to a modification. FIG. 18 illustrates an XY transverse section of the guide wire 100e at the same location as the XY transverse section of guide wire 100 illustrated in FIG. 4. FIG. 19 illustrates an XZ cross section of the guide wire 100e at the same location as the XZ cross section of the guide wire 100 illustrated in FIG. 5. Hereinafter, components of the guide wire 100e according to the modification, which are the same as the components of the guide wire 100 according to the first embodiment, are denoted with the same reference signs, thereby omitting the description thereof as appropriate.

In the guide wire 100e of the modification, a shape of a transverse section of a wire 40e is different from the shape of the transverse section of the wire 40 in the guide wire 100 of the first embodiment. In detail, as illustrated in FIG. 18, a transverse section of a base portion 40Ae is a perfect circle, similarly to the base portion 40A of the first embodiment. A surface of a folded portion 40Be facing the base portion 40Ae is a planar surface. In the present modification, an outer edge of a transverse section of the folded portion 40Be includes an arc portion APe and a straight portion SPe. The straight portion SPe is located at a position facing the base portion 40Ae on the outer edge of the folded portion 40Be. The arc portion APe is connected to both end portions of the straight portion SPe. That is, in this modification, the transverse section of the folded portion 40Be is partially circular. More specifically, the transverse section of the folded portion 40Be is a circular segment. The transverse section of the folded portion 40Be is a flattened circular segment. As illustrated in FIG. 19, a transverse section of a loop portion 40Ce includes an arc portion APe and a straight portion SPe. That is, in this modification, the transverse section of the loop portion 40Ce is partially circular. More specifically, the transverse section of the loop portion 40Ce is a circular segment. The transverse section of the folded portion 40Be is a flattened circular segment. As in this modification, the transverse section of the second portion may be partially circular.

The configuration of the guide wire 100 according to the above-described embodiment is merely an example and may be modified in various ways. For example, the guide wire may not include at least one of the first coating 31, the second coating 32, and the third coating 33.

The material for each member according to the above-described embodiments is merely an example and may be variously modified. The method of manufacturing the guide wire 100 according to each of the above-described embodiments is merely an example and can be modified in various ways. The treatment method using the guide wire 100 in the above-described embodiment is merely an example, and can be modified in various ways.

In the above-described embodiment, the guide wire 100 for treating a lesion in a blood vessel has been described as an example. The technology disclosed in this specification is similarly applicable to medical devices in general for treating lesions in a living body lumen.

Each of all the features described in each of the embodiments described above may be appropriately combined with another embodiment, or may be appropriately combined with a modification. Each of all the features described in each of the modifications described above may be appropriately combined with the embodiment or may be appropriately combined with another modification. Each of all the features described in each of the embodiments described above may be omitted as appropriate. Each of all the features described in each of the modifications described above may be omitted as appropriate.

1. A medical device comprising:
an elongated main body part; and
a leading portion connected to a distal end of the main body part and configured to enter a lesion, wherein
the main body part and the leading portion comprise a wire,
the main body part includes:
a first portion of the wire, and
a second portion of the wire that is different from the first portion, the second portion having a portion facing the first portion in a first direction intersecting an axial direction of the main body part,
the leading portion includes a third portion of the wire that is different from the first portion and the second portion and is located between the first portion and the second portion of the wire,
a surface of the first portion facing the second portion is a curved surface, and
a surface of the second portion facing the first portion is a planar surface.

2. The medical device according to claim 1, wherein
a transverse section of the first portion has a circular shape.

3. The medical device according to claim 1, wherein
an outer edge of a transverse section of the first portion includes an arc portion and a straight portion, and
the arc portion faces the second portion.

4. The medical device according to claim 3, wherein
the transverse section of the first portion has a partial circular shape.

5. The medical device according to claim 1, wherein
a transverse section of the second portion has a rectangular shape.

6. The medical device according to claim 1, wherein
an outer edge of a transverse section of the second portion includes an arc portion and a straight portion, and
the straight portion faces the first portion.

7. The medical device according to claim 6, wherein
the transverse section of the second portion has a partial circular shape.

8. The medical device according to claim 1, wherein
a transverse section of the third portion has a circular shape.

9. The medical device according to claim 1, wherein
a transverse section of the third portion has a rectangular shape.

10. The medical device according to claim 1, wherein
a shape of a transverse section of the first portion, a shape of a transverse section of the second portion, and a shape of a transverse section of the third portion are not similar to one another.

11. The medical device according to claim 1, wherein
a shape of a transverse section of the first portion and a shape of a transverse section of the third portion are similar to each other.

12. The medical device according to claim 1, wherein
a shape of a transverse section of the second portion and a shape of a transverse section of the third portion are similar to each other.

13. The medical device according to claim 1, wherein
a proximal end of the first portion is located at a proximal end portion of the main body part.

14. The medical device according to claim 13, wherein
a width of a distal end portion of the first portion in the first direction is greater than a width of a distal end portion of the second portion in the first direction.

15. The medical device according to claim 1, wherein
the main body part further includes a tubular body that covers the first portion and the second portion, and
the first portion and the second portion are joined to the tubular body.

16. The medical device according to claim 1, wherein
a proximal end of the first portion is located on a proximal end side with respect to a proximal end of the second portion.
